(19) United States
(12) Patent Application Publication
(10) Pub. No.: US 2026/0053976 A1
(43) Pub. Date: Feb. 26, 2026

(54) METHACRYLATED METHYLCELLULOSE AND OXIDIZED CARBOXYMETHYLCELLULOSE ADHESIVE HYDROGEL

(71) Applicant: Research Foundation of the City University of New York, New York, NY (US)

(72) Inventors: Steven B. Nicoll, Cedarhurst, NY (US); Jesse A. Martin, New York, NY (US); David J. Alpert, Brooklyn, NY (US)

(21) Appl. No.: 19/104,811

(22) PCT Filed: Aug. 21, 2023
(86) PCT No.: PCT/US23/72581

§ 371 Date: Feb. 19, 2025

Related U.S. Application Data
(60) Provisional application No. 63/399,279, filed on Aug. 19, 2022.

(51) Int. Cl. A61L 24/08; A61L 24/00; A61L 24/02

(52) U.S. Cl. CPC A61L 24/08; A61L 24/001; A61L 24/0031; A61L 24/02

(57) ABSTRACT

A biocompatible adhesive suitable for both internal and topical use. The adhesive is a tissue-adhesive hydrogel formed from an oxidized carboxymethylcellulose (oCMC) with aldehyde functional groups and a methacrylated methylcellulose (mMC).

### CROSS-REFERENCE TO RELATED APPLICATIONS

This application claims priority to, and is a non-provisional of, U.S. Patent Application 63/399,279 (filed Aug. 19, 2022) the entirety of which is incorporated herein by reference.

### STATEMENT OF FEDERALLY SPONSORED RESEARCH OR DEVELOPMENT

This invention was made with Government support under grant numbers 1701120 and 2214012 awarded by the National Science Foundation. The government has certain rights in the invention.

### BACKGROUND OF THE INVENTION

The subject matter disclosed herein relates to biocompatible adhesives that are suitable for both internal and topical use. There is a need for surgical adhesives that are safe for internal applications and stable. Synthetic cyanoacrylate glues (e.g., HISTOACRYL®, DERMABOND®) offer a high bonding strength, and can work in wet environments, but demonstrate high cell toxicity, low tensile strength, and patients often report a burning sensation after application. For these reasons, such bioadhesives are largely only approved for topical use. Tissue adhesives based on photopolymerizable poly (ethylene glycol) (PEG) hydrogels (e.g., FOCALSEAL™) are less prone to the aforementioned biocompatibility issues. However, they require ultraviolet (UV) illumination and long curing times, and generally exhibit inherently low internal strength. Even those adhesives that can be adapted for in situ gelation have the tendency to grow more brittle over time due to crystallization. Natural fibrin glues (e.g., TISSUCOL™, BERIPLAST™, BOLHEAL™ and BIOCOL™) present the risk of disease transmission due to the source material and, as they are protein-based, are highly degradable in the body due to enzymatic activity, limiting them to short-term applications. Alternatively, BIOGLUE® (bovine serum albumin and glutaraldehyde) is associated with toxicity and long setting times, while still remaining vulnerable to biodegradation if applied internally. Thus, current options for biological glues are inadequate for long-term internal wound closure. An improved biocompatible adhesive is therefore desired.

The discussion above is merely provided for general background information and is not intended to be used as an aid in determining the scope of the claimed subject matter.

### SUMMARY

A biocompatible adhesive suitable for both internal and topical use. The adhesive is a tissue-adhesive hydrogel formed from an oxidized carboxymethylcellulose (oCMC) with aldehyde functional groups and a methacrylated methylcellulose (mMC).

The technical problem to be solved is the creation of a biocompatible adhesive that adheres to biological tissue. The adhesive should resist degradation under physiological conditions and rapidly (e.g. within 4-15 min) form a hydrogel.

In a first embodiment, a composition of matter is provided. The composition of matter comprising a mixture of: an oxidized carboxymethylcellulose (oCMC) with aldehyde functional groups; and a methacrylated methylcellulose (mMC).

In a second embodiment, a composition of matter is provided. The composition of matter comprising a mixture of: an oxidized carboxymethylcellulose (oCMC) with aldehyde functional groups, wherein the oxidized carboxymethylcellulose has an initial molecular weight between 650 kDa and 750 kDa; a methacrylated methylcellulose (mMC) with an initial molecular weight between 30 kDa and 50 kDa; and an aqueous solvent selected from a group consisting of an aqueous buffer, an aqueous saline solution and water wherein the oxidized carboxymethylcellulose and the methacrylated methylcellulose are present in the aqueous solvent at a total concentration between 6-15% (w/v).

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic depiction of the formation of a tissue-adhesive hydrogel from oxidized carboxymethylcellulose (oCMC) mixed with a methacrylated methylcellulose (mMC).

FIG. 2 schematically depicts the formation of methacrylated methylcellulose (mMC) from methylcellulose (MC).

FIG. 3 schematically depicts the formation of oxidized carboxymethylcellulose (oCMC) from carboxymethylcellulose (CMC).

FIG. 4 is a graph that correlates a theoretical degree of oxidation (tDO) with a measured aldehyde modification percentage (DA).

FIG. 5A, FIG. 5B and FIG. 5C are graphs depicting rheological profiles for one tissue-adhesive hydrogel composition which formed the hydrogel via thermal gelation.

FIG. 6A, FIG. 6B and FIG. 6C are graphs depicting rheological profiles for one tissue-adhesive hydrogel composition which formed the hydrogel via redox-initiated gelation.

FIG. 7 depicts a graph showing adhesive strength of various tissue-adhesive hydrogel compositions to porcine skin.

FIG. 8 is a graph depicting Young's modulus for three tissue-adhesive hydrogel compositions.

FIG. 9A, FIG. 9B, FIG. 9C and FIG. 9D are graphs depicting stability profiles for one tissue-adhesive hydrogel.

FIG. 10A is a graph showing the results of lap shear testing on hydrogels with various concentration of mMC.

FIG. 10B and FIG. 10C are graphs of adhesive strength and strain, respectively, of various hydrogels with different mMC concentrations with mMC (3%) and mMC (4%) as controls.

FIG. 11A is a graph showing the results of lap shear testing on hydrogels with various ratios of oCMC:mMC.

FIG. 11B and FIG. 11C are graphs of adhesive strength and strain, respectively, of various hydrogels with different ratios of oCMC:mMC.

FIG. 12 illustrates a graph of DNA content for one tissue-adhesive hydrogel composition in comparison to a MC control and a gel-free control.

FIG. 13 illustrates a graph of DNA content for one tissue-adhesive hydrogel composition in comparison to a MC control and a gel-free control.

### DETAILED DESCRIPTION OF THE INVENTION

This disclosure provides a biocompatible adhesive that is based on biopolymers derived from a plant polysaccharide, cellulose, for engineering injectable, thermosensitive, tissue-adhesive hydrogels. More specifically, the biocompatible adhesive combines a modified form of methylcellulose (MC) and carboxymethylcellulose (CMC) in proportions refined to yield a semi-interpenetrating polymer network (sIPN) that can achieve in situ gelation and tissue adhesivity, while retaining the thermogelling nature of the methylcellulose. The disclosed tissue-adhesive hydrogels are useful in a wide variety of applications such as surgical glue for dermal tissues, internal wound closure, sealants for intervertebral disc herniations, as a hemostatic device and as an embolic agent.

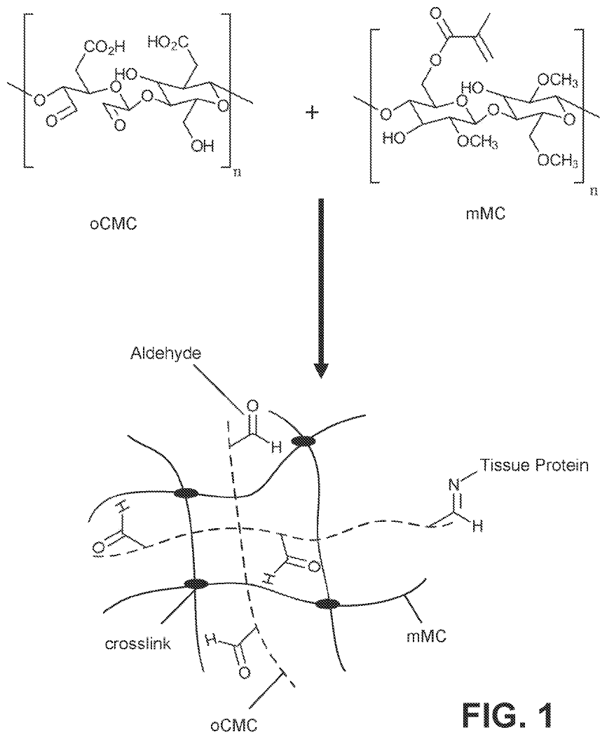

Referring to FIG. 1, an oxidized carboxymethylcellulose (oCMC) is mixed with a methacrylated methylcellulose (mMC). Under the reaction conditions described in this disclosure, the mMC crosslinks to form a polymer network. At temperatures above 33° C., increased hydrophobic interactions between the methoxy side groups on the mMC polymer backbone render the mMC relatively hydrophobic, and permits thermal gelation. The oCMC physically entangles within the polymer network formed by the mMC.

The oCMC is a relatively hydrophilic polysaccharide due to its carboxyl groups, which are ionized at physiologic pH (i.e. pH 7.40±0.05). Consequently, this attraction for water creates an environment that is more conducive to swelling, nutrient transport, and extracellular matrix (ECM) deposition in comparison to inert polymers, e.g., polyethylene glycol (PEG). Without wishing to be bound to any particular theory, ring-opening oxidation of C—C bonds along the pyranose rings of the CMC backbone (specifically at the C2-C3 bond) may impart tissue adhesivity via the formation of aldehydes, which can bind to proteins at the gel-tissue interface. This adhesivity affords resistance to implant movement and malpositioning. Because the constituent polysaccharides can only be cleaved by cellulase (an enzyme absent in humans), the covalently crosslinked cellulosic sIPN gels are not susceptible to enzymatic degradation in humans. Moreover, the interchain ester crosslinks between methacrylate moieties on the hydrophobic mMC network, which give the polymerized sIPN hydrogels mechanical stability, may be gradually hydrolyzed in a very limited manner, providing improved stability.

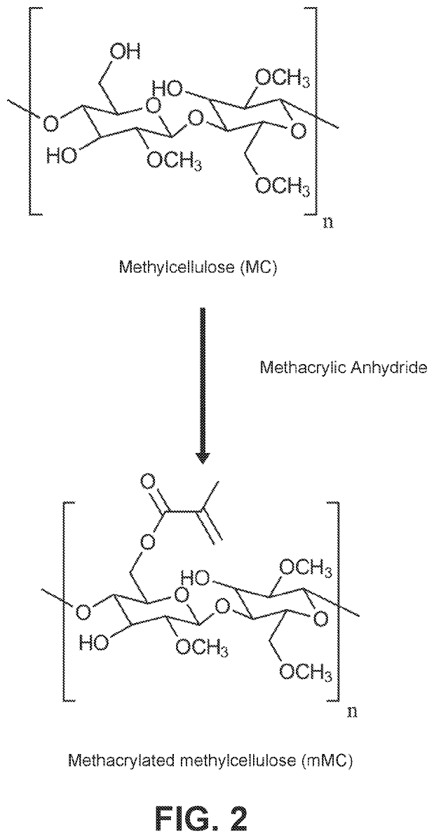

Referring to FIG. 2, the methacrylated methylcellulose (mMC) may be formed by treating methylcellulose (MC) with methacrylic anhydride. For example, a 1% w/v solution of MC (Sigma-Aldrich) is reacted with an excess (e.g. 20-40 fold excess) of methacrylic anhydride (Sigma-Aldrich) (target degree of methacrylation (DM) of 50%) in deionized water over 24 h at 4° C., and a pH of 8.0-8.5. The resulting solution is purified via membrane dialysis (Spectra/Por 1, MWCO 6-8 kDa) for 3-4 days against deionized water to remove excess, unreacted methacrylic anhydride. The purified mMC is recovered via lyophilization and the solid product is stored at −20° C. After acid hydrolysis of the purified mMC, the DM is confirmed via ¹H-NMR spectroscopy (500 MHz, Varian Unity Innova 500, Agilent Technologies). In one example, the DM was found to be about 6.09 mole % but can typically range from 2-10 mole %. In one embodiment, the DM is between 2-8%. In another embodiment, the DM is between 3-7 mole %. In another embodiment, the DM is between 5-7 mole %. The DM is determined by quantifying and normalizing the area under the peak of the methacrylate group (3 protons) divided by the area under the peak for the MC backbone (12 protons) measured from the NMR spectra. As such, the area under the peak of the methacrylate group was divided by 3 and the area under the peak for the MC backbone was divided by 12. In another embodiment, the DM is between 6-7 mole %. In another embodiment, the DM is between 8-10 mole %. In another embodiment, the DM is between 6-10 mole %.

The MC may be a medium viscosity MC with an initial molecular weight above 30 kDa and below 50 kDa (weight average). In another embodiment, the initial molecular weight is above 40 kDa and below 45 kDa. In another embodiment, the initial molecular weight is 41 kDa. The initial molecular weight refers to the molecular weight prior to mixing with the oCMC and initiators.

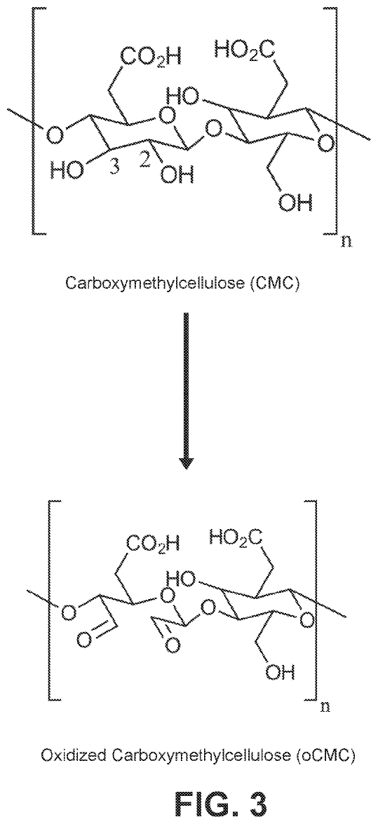

Referring to FIG. 3, oxidized carboxymethylcellulose (oCMC) is produced from carboxymethylcellulose (CMC) via oxidation. For example, a 1% w/v CMC (Sigma-Aldrich) solution is prepared in deionized water and reacted in a 1:1 molar ratio to the mass of CMC repeating units (reaction CMC concentration about 0.75% w/v and theoretical degree of oxidation (tDO) of 100%) with sodium periodate (NaIO₄) at about 35° C. for 4 hours with continual stirring, protected from light, and maintaining a pH of about 3. The oxidation reaction is quenched with ethanol at a 1:1 molar ratio with NaIO₄ in the solution, and the solution is subsequently purified via membrane dialysis (Spectra/Por 1, MWCO 25 kDa) for 3-4 days, recovered via lyophilization, and stored at-20° C. Some of the aldehydes are consumed in side-reactions (e.g. hemiacetal formation) so the actual degree of oxidation (DO) differs from the theoretical degree of oxidation (tDO). If desired, the actual degree of oxidation (DO) can be quantified by NMR. Quantification of degree of aldehyde modification (DA) is conducted using the hydroxylamine hydrochloride titration assay (Zhao and Heindel, Pharmaceutical Research 8, 400-402, 1991). In one embodiment, the DA was measured to be about 8.02±2.24 mole % but can range from 2-15%. In one embodiment, the DA is between 2-10 mole %. In another embodiment, the DA is between 7-9 mole %. In another embodiment, the DA is between 2-4 mole %. In another embodiment, the DA is between 5-7 mole %. In another embodiment, the DA is between 8-10 mole %. The CMC may be a high viscosity CMC with an initial molecular weight above 650 kDa and below 750 kDa (weight average). In another embodiment, the CMC has an initial molecular weight between 680-720 kDa. In yet another embodiment, the CMC has an initial molecular weight of 700 kDa. The initial molecular weight refers to the molecular weight prior to mixing with the mMC and initiators.

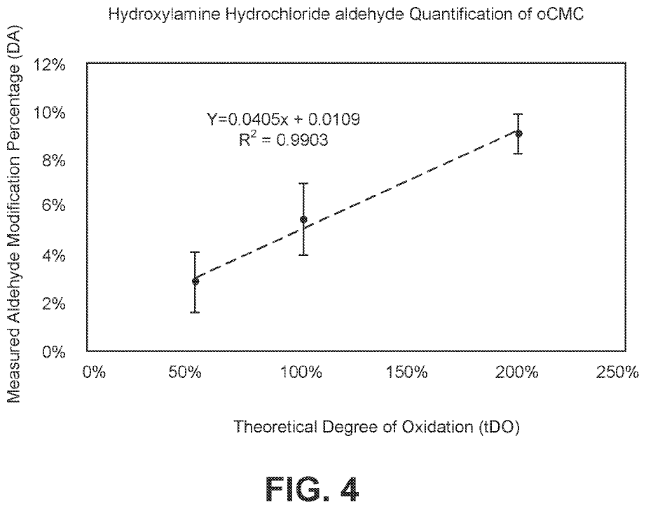

FIG. 4 is a graph depicting the measured degree of aldehyde modification percentage (DA) versus the theoretical degree of oxidation (tDO). By increasing or decreasing the amount of the oxidizing agent (e.g. sodium periodate) and reaction time one can control the DA.

In one embodiment, sIPN hydrogels are produced by gently pulling the mMC and oCMC into small (about 0.25-1.0 cm diameter) clusters of polymer fiber. While dry, these clusters are combined at specific mass ratios of oxidized CMC to methacrylated MC polymer (oCMC:mMC) as two identical aliquots and then stirred. The mass ratios vary between 0.5:1 and 4:1 (e.g. 1:2 (i.e. 0.5:1); 1:1; 2:1; 3:1, 4:1). The polymers are dissolved by the addition of a buffer (e.g. Dulbecco's phosphate-buffered saline (DPBS) (1×), PBS, 0.1 M CaCl₂), similar non-buffered saline solutions, water, etc.) at volumes appropriate for yielding the desired total concentration less the volume needed for redox polymerization initiators that are added later (generally 6-15% w/v). Dissolution is expedited by utilizing cycles of static cold storage (4-8° C., 12 hrs), manual mixing/homogenization, ice bath ultrasonication (BRANSON ULTRASONICS™ CPXH Series Ultrasonic Cleaning Bath, 40 MHz, about 10-20 mins, high power, 8-15° C.), and centrifugation (swinging bucket, about 5-10 mins, 5 krpm, 4° C.) every 1-2 days, wherein the goal is to eliminate heterogeneities in the sIPN prepolymer solution comprises mMC and oCMC. The dissolution time is about 8 days, and the limit of attainable and uniform bulk concentration is about 15% w/v.

Generally, the concentration of the mMC is between 3-5% w/v for the sIPN materials because lower values did not reliably adhere to tissue, and higher values were too difficult to inject, if not impossible. The oCMC is generally present in the buffer at a concentration between 2-10% (w/v) and the mMC is generally present in the buffer at a concentration between 3-5% (w/v).

Once each aliquot of the sIPN prepolymer solution is fully dissolved, polymerization initiators (e.g. ammonium persulfate (APS) as an oxidizing agent and ascorbic acid (AA) or N,N,N′,N′-tetramethylethylenediamine (TEMED) as a reducing agent/accelerator, all from Sigma-Aldrich) are separately added as solutions to one of the two aliquots. For example, APS may be added to the oCMC solution while AA may be added to the mMC solution. The amount of initiator is controlled such that the respective final initiator (generally 10-20 mM) and polymer concentrations are the same between aliquots. After addition of the initiator another cycle of expedited dissolution is performed. The solutions are then loaded in separate barrels of a dual-barrel syringe (Pac-Dent), centrifuged (fixed angle bucket, 5-10 mins, about 4.5 krpm, room temperature), and subsequently combined via extrusion through a mixing tip. This process allows the polymerization initiators to interact, crosslinking the mMC solution into a hydrogel via redox-initiated polymerization of the mMC methacrylate side groups. The APS/AA redox initiation system enables free radical polymerization without need for external polymerization initiation elements (e.g., UV light). Such a configuration is particularly useful for internal applications, such as injection into joints (e.g. ruptured intervertebral discs).

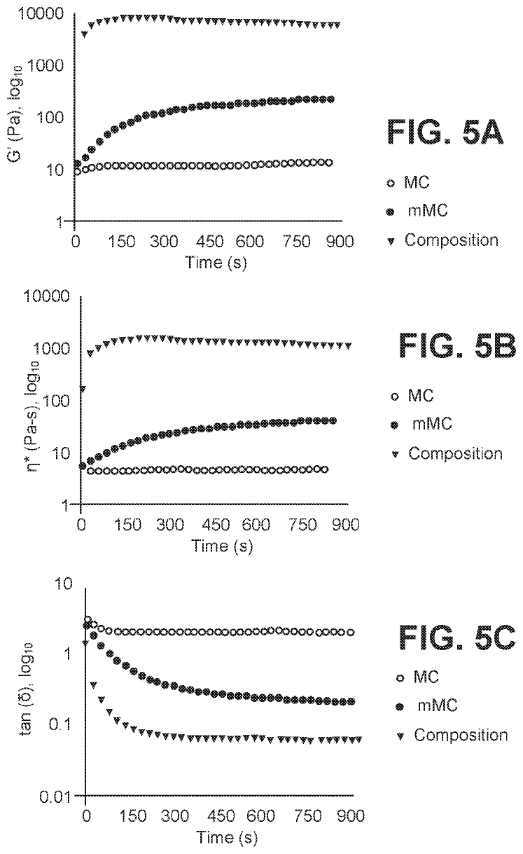

FIG. 5A, FIG. 5B and FIG. 5C are graphs depicting rheological profiles for one composition (mass ratio of oCMC:mMC of 2:1, 4% (w/v) mMC) which formed the hydrogel via thermal gelation. The oCMC had an initial molecular weight of 700 kDa and the mMC had an initial molecular weight of 41 kDa. DM was 3.6 mole %, DA was 8.02 mole % and the theoretical tDO was 100%. The oCMC and mMC were dissolved as described elsewhere in this disclosure but no polymerization initiators were added. The dissolved prepolymers were subjected to rheometry (AR2000, TA Instruments, 1.0% strain, 1 Hz) using a cone plate geometry (25 mm, 0.201 rad) while applying a temperature step (ramp rate: about 50° C./min from about 4° C. to 37° C.) and holding the temperature at 37° C. for 15 min. FIG. 5A depicts the resulting storage modulus (G′). FIG. 5B depicts the complex viscosity η*. FIG. 5C depicts the loss tangent (tan (δ)). Controls of 4% (w/v) MC and 4% (w/v) mMC were used.

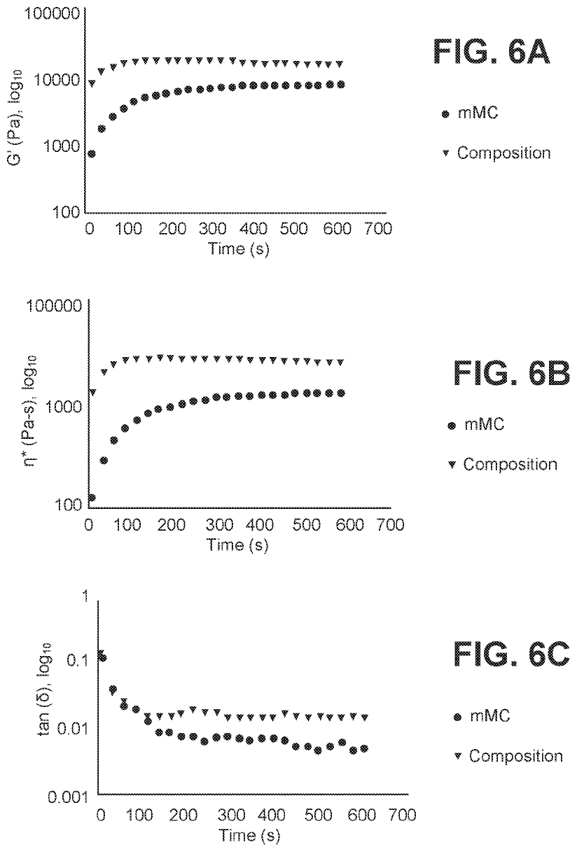

Likewise, FIG. 6A, FIG. 6B and FIG. 6C are graphs depicting rheological profiles for one composition (mass ratio of oCMC:mMC of 2:1, 4% (w/v) mMC) which formed the hydrogel via redox-initiated gelation. The oCMC had an initial molecular weight of 700 kDa and the mMC had an initial molecular weight of 41 kDa. DM was 3.6 mole %, DA was 8.02 mole % and the theoretical tDO was 100%. The oCMC and mMC were dissolved as described elsewhere in this disclosure. Polymerization initiators (ammonium persulfate (APS) and ascorbic acid (AA)) were used at 20 mM. The dissolved prepolymers were subjected to rheometry (AR2000, TA Instruments, 1.0% strain, 1 Hz) using a cone plate geometry (25 mm, 0.201 rad) while applying a temperature step (ramp rate: about 50° C./min from about 4° C. to 37° C.) and holding the temperature at 37° C. for 15 min. FIG. 6A depicts the resulting storage modulus (G′). FIG. 6B depicts the complex viscosity η*. FIG. 6C depicts the loss tangent (tan(δ)). A control of 4% (w/v) mMC was used.

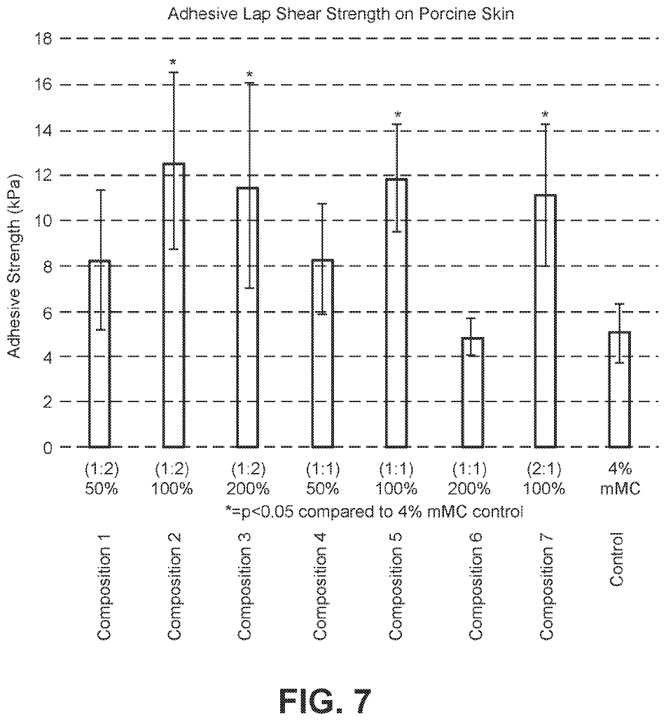

FIG. 7 is a graph that depicts adhesive strength for various hydrogels to defatted porcine skin via lap shear testing. The mass ratio of oCMC:mMC was varied as was the theoretical tDO. FIG. 4 correlates theoretical tDO to measured DA. Each hydrogel was formed from a 4% (w/v) mMC solution with a corresponding amount of oCMC as shown in Table 1. The CMC had a molecular weight of 700 kDa and the MC had a molecular weight of 41 kDa. The gels were applied to 8-mm biopsies of porcine skin set in glass molds. The gels were allowed to undergo redox-initiated polymerization using APS/AA (both at 20 mM) in an incubator at 37° C. for 30 minutes. The gels were then glued to strips of sandpaper with cyanoacrylate glue and tested in Bose Electroforce at 3 mm/min. The failure stresses observed for the disclosed hydrogel formulations ranged from about 8-20 kPa, comparable to those reported for similar commercially-available fibrin-based tissue adhesives, e.g., TISSEEL™. The most adhesive formulations showed an adhesive strength between 11-13 kPa. This demonstrates strong adhesive strength between the disclosed hydrogels and the porcine skin. In one embodiment, the adhesive strength is between 6 and 25 kPa. In another embodiment, the adhesive strength is between at least 10 kPa.

TABLE 1

| | oCMC:mMC mass | [oCMC] | | | |
|---|---|---|---|---|---|
| Composition | ratio | (% w/v) | tDO | DA | DM |
| Composition 1 | 1:2 | 2 | 50 | 2.9% | 6.09% |
| Composition 2 | 1:2 | 2 | 100 | 5.5% | 6.09% |
| Composition 3 | 1:2 | 2 | 200 | 9.1% | 6.09% |
| Composition 4 | 1:1 | 4 | 50 | 2.9% | 6.09% |
| Composition 5 | 1:1 | 4 | 100 | 5.5% | 6.09% |
| Composition 6 | 1:1 | 4 | 200 | 9.1% | 6.09% |
| Composition 7 | 2:1 | 8 | 100 | 5.5% | 6.09% |

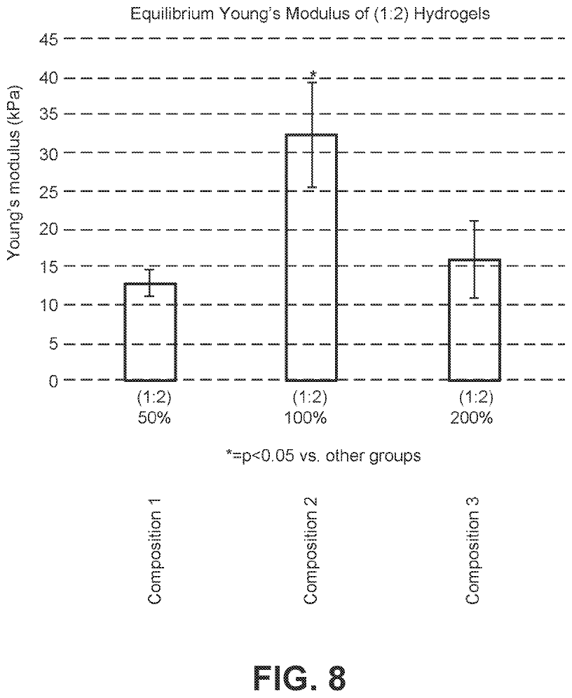

FIG. 8 is a graph that depicts the Young's Modulus of Composition 1, 2 and 3. Cylindrical gels (2-mm thick, 5-mm diameter) were cast in glass molds and allowed to undergo redox-polymerization at 37° C. for 30 minutes. The gels were placed in PBS and allowed to swell overnight. Unconfined compression was performed in an environmental chamber filled with PBS at room temperature (22° C.). A 30 min creep was performed with a load of 1g before a stress-relaxation test at 5, 10, and 15% strain. The equilibrium values were plotted, and the slope taken as the equilibrium Young's modulus. In one embodiment, the Young's modulus is between 10 and 75 kPa.

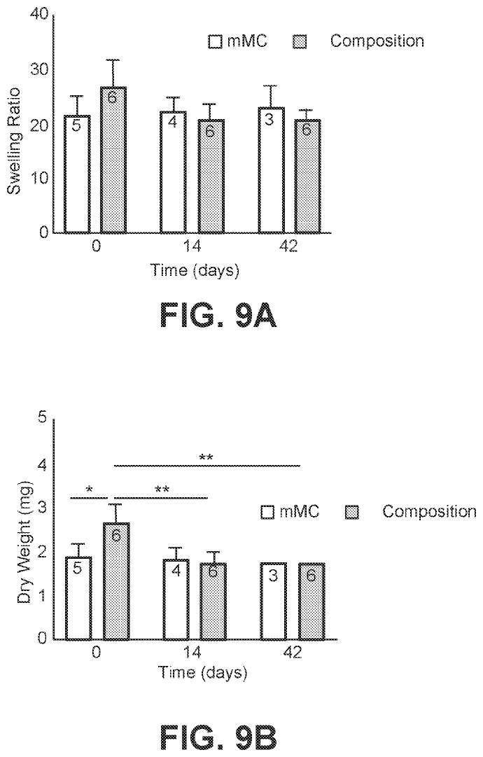

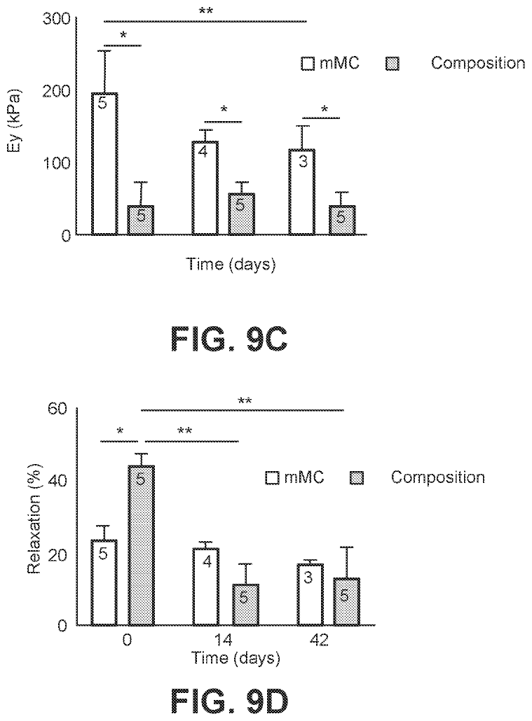

FIG. 9A, FIG. 9B, FIG. 9C and FIG. 9D are graphs depicting the swelling ratio, the dry weight, the equilibrium Young's modulus and the percentage relaxation, respectively, over time, for a composition wherein oCMC:mMC is 2:1, 4% (w/v) mMC) which formed the hydrogel via redox-initiated gelation. The oCMC had an initial molecular weight of 700 kDa and the mMC had an initial molecular weight of 41 kDa. DM was 3.6 mole %, DA was 8.02 mole % and the theoretical tDO was 100%. The composition was incubated in PBS at 37° C. for 42 days. Significant differences in properties are indicated by * with significant difference over time indicated by ** relative to a 4% mMC control. The dry weight changed significantly during the first two weeks but remained stable thereafter.

Rheological characterization of redox-initiated gelation for the composition (as in FIG. 6A-6C) indicated a more robust profile for the disclosed hydrogels, with significantly higher values of storage modulus (G′) and complex viscosity (η*) than 4% w/v mMC references. Both materials exhibited declines in tan (δ), the ratio of viscous to elastic response of the material, indicating an increase in elastic behavior.

Throughout these evaluations, the hydrogels demonstrated consistent gelation even when exposed to air during polymerization. This is in contrast with previous investigations wherein oxygen-mediated interference with the redox-initiated reactions promoted crosslinking and inhibited gelation. While at storage temperatures (about 4-10° C.), the disclosed solutions remained at a viscosity of less than 1200 Pa-s which is comparable to commercially available dermal fillers. As an example, the initial complex viscosity of Composition 1 was 77.23±42.89 Pa-s and that of the composition in FIG. 6A-6C was 1148.24±299.66 Pa-s.

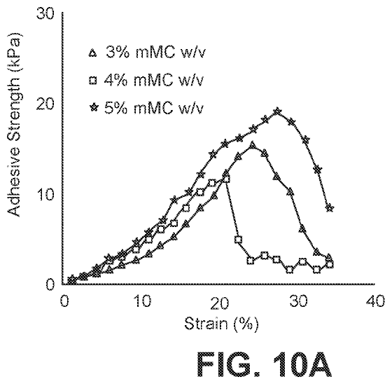

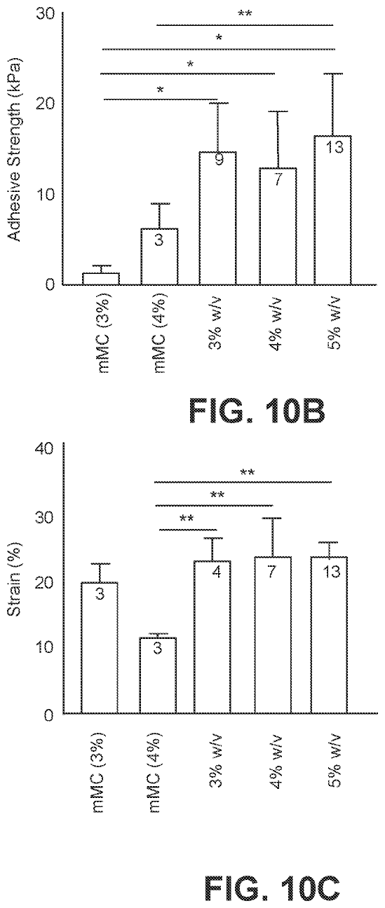

FIGS. 10A-C depict results of lap shear testing on hydrogel compositions with varied concentration of the mMC (3%, 4% and 5%, w/v). The oCMC:mMC was 2:1 with oCMC having an initial molecular weight of 700 kDa and the mMC having an initial molecular weight of 41 kDa. DM was 3.6 mole %, DA was 8.02±2.24 mole % and the theoretical tDO was 100%. In FIG. 10B and FIG. 10C, significant differences from mMC (3%) and mMC (4%) controls are indicated by * and **, respectively. FIG. 10A shows the adhesive strength (kPa) as a function of strain (%). FIG. 10B shows the adhesive strength (kPa) for different compositions. FIG. 10C shows the strain (%) for the same compositions.

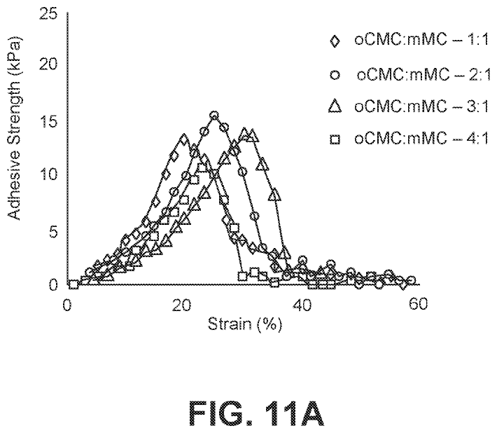

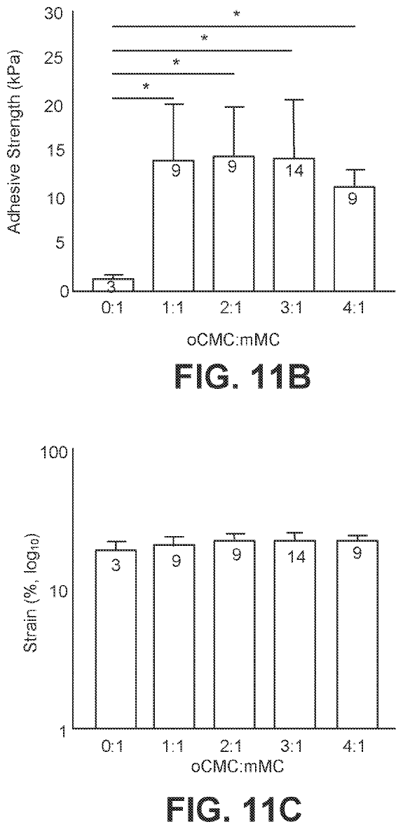

FIG. 11A-C depict results of lap shear testing on hydrogel compositions with 3% w/v mMC at various oCMC:mMC ratios. The oCMC had an initial molecular weight of 700 kDa and the mMC had an initial molecular weight of 41 kDa. The concentration of mMC was 3% w/v. DM was 3.6 mole %, DA was 8.02±2.24 mole % and the theoretical tDO was 100%. FIG. 11A shows a graph of adhesive strength (kPa) versus strain (%). FIG. 11B shows adhesive strength (kPa) for various ratios of oCMC:mMC. FIG. 11C shows strain for various ratios of oCMC:mMC. mMC (3%) was used as a control (oCMC:mMC=0:1) with significant differences from this control indicated by *.

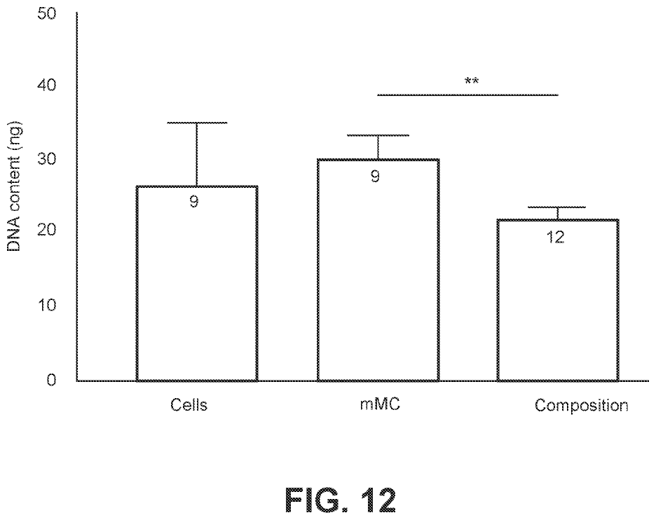

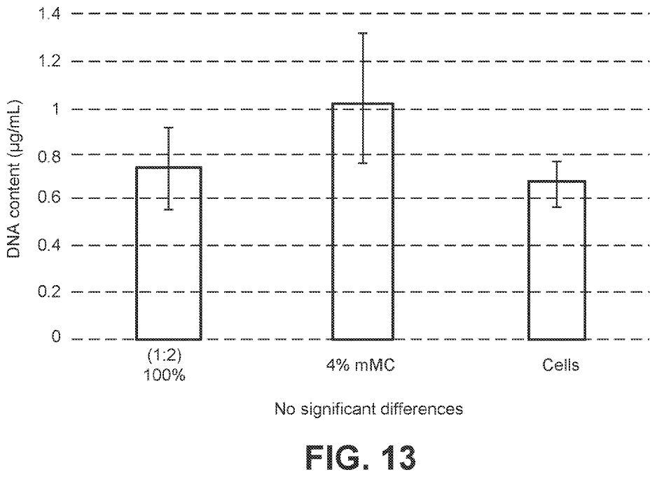

FIG. 12 is a graph showing the DNA content of bovine nucleus pulposus cells that were cultured in the presence of the disclosed hydrogels for 24 hours. A gel-free control (labeled “Cells”) was used. These studies indicated the hydrogels have negligible cytotoxicity. A gel-based control formed from 4% mMC was also used. Evaluation of cytocompatibility was performed in a 24-hr contact culture study (oCMC:mMC is 2:1, 4% (w/v) mMC, which formed the hydrogel via redox-initiated gelation, the oCMC had an initial molecular weight of 700 kDa, the mMC had an initial molecular weight of 41 kDa. DM was 3.6 mole %, DA was 8.02 mole % and the theoretical tDO was 100%) with confluent bovine nucleus pulposus (NP) cells at 37° C. and 5% CO₂, seeded in 12-well culture plates with high glucose Dulbecco's Modified Eagle Medium (Gibco) containing 1% Penicillin/Streptomycin (Gibco) and 10% fetal bovine serum (Gibco). Total DNA content was measured via the PicoGreen assay (Invitrogen, Thermo Fisher Scientific) to assess cell proliferation after 24 hours of exposure to gels, with calf thymus DNA (Sigma-Aldrich) used to create a standard curve. Calcein AM Live/Dead assay (Invitrogen, Thermo Fisher Scientific) was used to visually evaluate cell viability. Gel-free cultures and cultures containing gels composed solely of 4% w/v mMC were used as references. While the composition demonstrated a significantly lower DNA production relative to the mMC control (p<0.0049), the difference from gel-free references was not statistically significant (p<0.1771), as was the difference between gel-free references and the composition (p<0.2956). This was further supported by live/dead staining images (not shown) which showed a large population of viable cells with a healthy morphology, and relatively few dead cells. FIG. 13 depicts a corresponding graph for Composition 2. In FIG. 13, human dermal fibroblasts were cultured in the presence of Composition 2 and mMC gels for 24 hours. The DNA concentration was measured using the PICOGREEN™ dsDNA Assay (ThermoFisher). The label “Cells” refers to a gel-free control culture. Cells were plated at a density of 20,000 cells/ml and maintained in Dulbecco's Modified Eagle Medium with 10% fetal bovine serum and antibiotics.

### Example Hydrogel Formulation

A specific reaction mixture for a hydrogel (oCMC:mMC=1:2) with a concentration of 4% (w/v) mMC and 2% (w/v) oCMC were prepared as follows: In a first 50 ml conical tube, 30 mg of mMC was mixed with 0.675 mL of PBS buffer. In a second 50 ml conical tube, 15 mg of oCMC was mixed with 0.675 mL of PBS buffer. Both conical tubes were fixed to a vortexer on high speed for 30 min. Both conical tubes were spun down in a centrifuge at 5 krpm for 5 min. Over the next 5-7 days a metal spatula was used to manually mix the content of each tube for no more than 30-60s and then spun down at 5 krpm.

To ready the polymer for gelation, initiators ammonium persulfate (APS) and ascorbic acid (AA) were prepared the same day. 45 mg of APS was combined with 0.986 mL of PBS, and 35 mg of AA was combined with 0.994 mL of PBS to create two 200 mM solutions of each initiator, respectively. To the first conical tube (the mMC solution) 0.075 mL of the APS solution was added. To the second conical tube (the oCMC solution) 0.075 mL of the AA solution was added. This diluted the initiator concentration to 20 mM. In other embodiments, each initiator is present at a concentration between 10-20 mM. Each tube was then manually mixed and spun down at 5 krpm twice before loading the mixtures into the individual barrels of a dual barrel syringe. The syringe is spun down at 4 krpm to remove bubbles from the polymer solutions. The polymer mixture is now ready for extrusion through the mixing tip, which will combine the initiators and begin polymerization. A stable hydrogel forms within 10-15 minutes of mixing. These gelation times were in the range of the surgical ISO standard 5833/1-1999 E for injectable materials (4-15 min), which is a distinct advantage over other in situ gelling systems that require several hours to achieve maximum mechanical properties.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter comprising a mixture of:
an oxidized carboxymethylcellulose (oCMC) with aldehyde functional groups; and
a methacrylated methylcellulose (mMC).

2. The composition of matter as recited in claim 1, wherein the methacrylated methylcellulose (mMC) has an initial molecular weight between 30 kDa and 50 kDa.

3. The composition of matter as recited in claim 1, wherein the oxidized carboxymethylcellulose (oCMC) has an initial molecular weight between 650 kDa and 750 kDa.

4. The composition of matter as recited in claim 1, wherein the methacrylated methylcellulose (mMC) has an initial molecular weight of 41 kDa.

5. The composition of matter as recited in claim 1, wherein the oxidized carboxymethylcellulose (oCMC) has an initial molecular weight of 700 kDa.

6. The composition of matter as recited in claim 1, wherein the methacrylated methylcellulose (mMC) is modified with methacrylate groups at a degree of methacrylation (DM) of 2-10%.

7. The composition of matter as recited in claim 1, wherein the methacrylated methylcellulose (mMC) is modified with methacrylate groups at a degree of methacrylation (DM) of 3-7%.

8. The composition of matter as recited in claim 1, wherein the oxidized carboxymethylcellulose (oCMC) is modified with aldehyde groups at a degree of aldehyde modification (DA) of 2-15%.

9. The composition of matter as recited in claim 1, wherein the oxidized carboxymethylcellulose (oCMC) and the methacrylated methylcellulose (mMC) are present at a weight ratio (oCMC:mMC) between 0.5:1 and 4:1.

10. The composition of matter as recited in claim 1, wherein the mixture consists of the oxidized carboxymethylcellulose (oCMC), the methacrylated methylcellulose (mMC), a redox initiator and an aqueous solvent selected from a group consisting of an aqueous buffer, an aqueous saline solution and water.

11. The composition of matter as recited in claim 10, wherein the redox initiator comprises ammonium persulfate and ascorbic acid.

12. The composition of matter as recited in claim 11, wherein the ammonium persulfate is present at a concentration between 10-20 mM and the ascorbic acid is present at a concentration between 10-20 mM.

13. The composition of matter as recited in claim 10, wherein the oxidized carboxymethylcellulose (oCMC) is present in a concentration between 2-10% (w/v) and the methacrylated methylcellulose (mMC) is present in a concentration between 3-5% (w/v).

14. The composition of matter as recited in claim 10, wherein the oxidized carboxymethylcellulose (oCMC) and the methacrylated methylcellulose (mMC) are collectively present in a total concentration between 6-15% (w/v).

15. A semi-interpenetrating, polymer network hydrogel formed from the composition of matter as recited in claim 1.

16. The hydrogel as recited in claim 15, wherein the semi-interpenetrating polymer network hydrogel has an adhesive strength of between 6 and 25 kPa.

17. The hydrogel as recited in claim 15, wherein the semi-interpenetrating polymer network hydrogel has a compressive equilibrium Young's modulus between 10 and 75 kPa.

18. A composition of matter comprising a mixture of:
an oxidized carboxymethylcellulose (oCMC) with aldehyde functional groups, wherein the oxidized carboxymethylcellulose has an initial molecular weight between 650 kDa and 750 kDa;
a methacrylated methylcellulose (mMC) with an initial molecular weight between 30 kDa and 50 kDa; and
an aqueous solvent selected from a group consisting of an aqueous buffer, an aqueous saline solution and water wherein the oxidized carboxymethylcellulose and the methacrylated methylcellulose are present in the aqueous solvent at a total concentration between 6-15% (w/v).
